(19) United States
(12) Patent Application Publication
(10) Pub. No.: US 2011/0151516 A1
(43) Pub. Date: Jun. 23, 2011

(54) PROCESS FOR THE EXTRACTION OF SUGARS AND LIGNIN FROM SOLID BIOMASS

(76) Inventors: Evert VAN DER HEIDE, Amsterdam (NL); Xuebing Zhao, (US)

(21) Appl. No.: 12/972,111

(22) Filed: Dec. 17, 2010

(30) Foreign Application Priority Data
Dec. 18, 2009 (EP) 09179842.1

(51) Int. Cl. C12P 19/00; C08H 7/00; C12P 1/00

(52) U.S. Cl. 435/72; 530/500; 435/41

(57) ABSTRACT

A process for the extraction of sugars and/or lignin from lignocellulose-containing solid biomass is provided.

This application claims the benefit of European Application No. 09179842.1 filed Dec. 18, 2009 which is incorporated herein by reference.

### FIELD OF THE INVENTION

The invention provides a process for the extraction of hemicellulose (sugars) and lignin from lignocellulose-containing solid biomass.

### BACKGROUND OF THE INVENTION

Ethanol is presently produced by fermentation of sugars derived from corn starch or sugar cane. There is, however, a strong drive to produce ethanol from lignocellulosic biomass, also referred to as cellulosic biomass, such as agricultural wastes, grasses, forestry wastes, and sugar processing residues. Before converting lignocellulosic biomass into sugars that can be fermented into ethanol, it is desired to first pre-treat the lignocellulosic biomass to make the cellulose, and optionally also the hemicellulose, digestible for enzymes that are able to depolymerise the cellulose and hemicellulose into their monomeric sugars.

Biomass pre-treatment processes that improve enzymatic digestibility are known in the art. Currently applied pre-treatment methods usually involve steam explosion and/or dilute acid pre-hydrolysis.

In U.S. Pat. No. 4,461,648 for example, steam explosion of lignocellulosic feedstock in the presence of sulphuric acid is described.

For the production of cellulose in pulp and paper production, cellulosic biomass can be heated in a solvent comprising formic acid.

In WO-A-97/26403 for example, a process is described for producing pulp from reed canary grass by subjecting the grass to a formic acid cooking step, optionally followed by a bleaching step.

In U.S. Pat. No. 6,139,683, a similar process using acetic acid is described. U.S. Pat. No. 6,139,683 describes a process for the extraction of cellulose from lignocellulose, which extraction is carried out by means of heating with an aqueous acetic acid and formic acid under pressure. The obtained cellulose still has a low residual lignin content. The cellulose can be subsequently be bleached with ozone and peracetic acid to high grades of white. In the examples cellulose was bleached with concentrations up to 1.3% peracetic acid solution in water and up to 0.7 wt % peracetic acid in acetic acid.

EP0508064 describes a three stage process for delignification of cellulose-containing raw materials, wherein pulping is initially performed with a solution of concentrated aqueous acetic acid at an elevated temperature under an elevated pressure. The resultant acetic acid-moist pulp is treated in a second stage with the same solution under addition of nitric acid and is then washed or extracted with water or with pulping solution. In the third treatment stage the acetic acid-moist pulp is treated with an ozone containing gas. After the above process a final bleaching can be carried out using for example peracetic acid. In the examples final bleaching was carried out with a solution containing about 0.06% peracetic acid, which solution was made up out of 385.2 gram water, 40 ml of sodium carbonate solution and 0.65 ml of 40% peracetic acid solution.

The use of oxidative processes for paper pulping is described for instance by Ricketts J. Drew, “Role of peracids revisited for delignification, bleaching”, Pulp & Paper, 1995, 69(3): 89-94. However, these processes use the peracids, and/or other oxidative processes for the bleaching of pulp for use in paper.

An important disadvantage of the prior art processes is that lignin is not sufficiently removed from the lignocellulosic biomass. The lignin that remains present in the cellulose has a negative effect on an enzymatic hydrolysis, and can give higher handling costs in downstream processing, in particular in the distillation of ethanol from a fermented sugar stream.

In addition, the lignin removed in the prior art processes has a low quality and usually can only serve as fuel.

### SUMMARY OF THE INVENTION

A process for the extraction of sugars and/or lignin from lignocellulose-containing solid biomass is provided comprising:

(a) contacting the lignocellulose-containing solid biomass with a pulping solution comprising at least 5 to 95% by weight of acetic acid at a temperature in the range of from 80 to 195° C. thereby producing a first mixture;
(b) separating the first mixture obtained in (a) into a pre-treated solid residue and a liquid stream comprising dissolved hemicellulose;
(c) contacting the pre-treated solid residue with a pulping solution comprising at least 5 to 95% by weight of peracetic acid at a temperature in the range of from 50 to 95° C. thereby producing a second mixture; and
(d) separating the second mixture obtained in (c) into a solid residue comprising delignified cellulose, and a liquid stream comprising dissolved lignin.

### DETAILED DESCRIPTION OF THE INVENTION

It has now been found that solid lignocellulose-containing biomass can be pre-treated by subjecting it to an extraction with a pulping medium comprising acetic acid at a relatively mild temperature to remove hemi-cellulose, followed by a second extraction and oxidative treatment with acetic acid peroxide (further referred to as peracetic acid yielding a delignified cellulose fraction that is suitable for enzymatic hydrolysis, as well as a lignin fraction that can be directly used for the preparation of phenolic formaldehyde resins.

Furthermore, non-wood derived lignocellulose, such as corn-stover, bagasse, and other grasses such as sweet sorghum, miscanthus and bamboo can be treated by the subject process as the silica present in the biomass is not dissolved during the process, but remains as a solid residue that is discarded as part of the product. This is particular advantageous as compared to process routes that employ alkaline treatment steps, such as the Kraft process.

Accordingly, the process for the extraction of sugars and/or lignin from lignocellulose-comprising solid biomass comprises:

(a) contacting the lignocellulose-comprising solid biomass with a pulping solution comprising at least 5 to 95% by weight of acetic acid at a temperature in the range of from 80 to 195° C.,
(b) separating the mixture obtained in (a) into a pre-treated solid residue and a liquid stream comprising dissolved hemicelluloses,
(c) contacting the pre-treated solid residue with a pulping solution comprising at least 5 to 95% by weight of peracetic acid at a temperature in the range of from 50 to 95° C.,
(d) separating the mixture obtained in (c) into a solid residue comprising delignified cellulose, and a liquid stream comprising dissolved lignin, and
(e) optionally precipitating lignin from the liquid stream obtained in step (d).

The process advantageously produce cellulose that can be converted into paper that requires less bleaching.

In addition the produced cellulose is suitable for enzymatic hydrolysis into sugars, which sugars in turn can be fermented into alcohols such as ethanol and/or hydrocarbons.

In another embodiment, a process for converting lignocellulose into sugars and lignin is provided, the process comprising a pre-treatment step, a washing step, an enzymatic hydrolysis step and a lignin precipitation step. The process is particularly suitable for preparing sugars from biomass for fermentation into bio-ethanol, as well as high quality lignin that can be directly used for lignin thermosetting resins.

The process according to the present invention allows to separate lignocellulose into cellulose, hemi-cellulose (and its breakdown products) and high quality lignin. In particular the lignin may be directly employed to form thermosetting resin products with carbonyl compounds such as formaldehyde, through the phenolic hydroxyl-groups and active sites on the aromatic ring.

The present process thus permits to not only convert the cellulose and hemicellulose into valuable intermediates or final products are used, but also produces lignin can be employed as chemical intermediate rather than as fuel only.

In step (a), lignocellulose is contacted with a pulping solution. The pulping solution preferably comprises at least 50 wt % acetic acid. The biomass to pulping solution ratio preferably is in the range of from 1:3 to 1:50.

Preferably, the solution contains acetic acid, water and optionally recycled liquid stream(s) obtained from step (b) and/or (d). The liquid stream(s) from step (b) and/or (d) are herein also referred to as black liquor.

This may be done in any reactor or other means suitable for the reaction and capable of operating under the conditions of step (a).

The solution may further contain water and/or organic compounds known to be suitable organic solutions for organosols processes. Examples of such solvents or acids are methanol, ethanol, acetone, ethylene glycol, triethylene glycol, tetrahydrofurfuryl alcohol, formic acid, oxalic acid, acetylsalicylic or salicylic acid.

Preferably, the solution contains less than 20 wt. %, more preferably less than 10 wt. % of organic components other than acetic acid.

Substantially pure acetic acid or mixtures essentially consisting of acetic acid and water are particularly preferred solutions. Reference herein to substantially pure acetic acid is to a solution containing at least 98 vol % acetic acid. In case of an acetic acid/water mixture as solution, the mixture preferably contains in the range of from 50 to 95 vol % acetic acid.

Preferably, the solution further contains a small amount of an inorganic acids such as sulphuric acid, phosphoric acid or hydrochloric acid that will act as a catalyst.

In pre-treatment step (a), the biomass is contacted with the pulping solution at a temperature in the range of from 80 to 195° C., preferably of from 100 to 170° C., more preferably of from 105 to 150° C.

Preferably, pretreatment step (a) is carried out at a pressure in the range of from 1 to 10 bar, more preferably of from 1 to 3 bar. Pre-treatment at atmospheric pressure is particularly preferred.

Prior to step (a), the solid biomass is typically comminuted into pieces or particles of a small size, preferably pieces or particles with an average length of less than 3 cm, more preferably an average length in the range of from 0.5 to 2.5 cm.

Any suitable lignocellulose-containing solid biomass may be used in the process according to the invention. Examples of suitable biomass are agricultural wastes such as corn stover, soybean stover, corn cobs, rice straw, rice hulls, oat hulls, corn fibre, cereal straw like wheat, barley, rye, and oat straw; grasses such as switch grass, miscanthus, cord grass, rye grass, reed canary grass; forestry biomass such as wood and saw dust; recycled paper or pulp fibres; sugar processing residues such as bagasse and beet pulp. Cereal straw, especially wheat straw, is particularly preferred.

In pre-treatment step (a), at least part of the hemicellulose is hydrolysed and removed from the solid biomass. Also part of the lignin is removed from the solid biomass. Thus, the cellulose is made more accessible for subsequent enzymatic hydrolysis thus improving its enzymatic digestibility.

The biomass will be contacted with the solution for a time period sufficient to achieve at least partial hemicellulose hydrolysis and/or partial delignification, typically in the range of from 0.5 to 10 hours, preferably in the range of from 1 to 5 hours.

The biomass-to-fresh pulping solution ratio is preferably in the range of from 1:3 to 1:50, preferably of from 1:5 to 1:20.

Pretreatment step (a) may be carried out in any reactor configuration suitable for solid-liquid contact, for example a co-current, counter-current of flow-through configuration.

In pretreatment step (a), suitably a mixture containing pre-treated solid residue and a liquid stream comprising dissolved hemicelluse is obtained.

In step (b), the mixture obtained in (a) is separated into a pre-treated solid residue and a liquid stream comprising dissolved hemicellulose. This separation can be carried out by any suitable technique known in the art, for example filtration or centrifugation. The separation in step (b) preferably is performed by pressing the mixture in a suitable press filter, screw press or any other means suitable to separate a solid/liquid mixture that is highly corrosive and highly viscous at the reaction conditions. Examples for suitable means include conveyer belts, screw presses, extruders, centrifuges. Preferably a screw press is used.

Steps (a) and (b) may be integrated, preferably when operating the process continuously. Both steps may happen simultaneously, e.g. in a screw press. The liquid stream(s) obtained in step (b) are referred to herein as washing black liquor.

In order to remove remaining solvent and lignin, step (b) further may comprise one or more optional washing steps with a washing liquid such as water, or otherwise suitable aqueous recycle stream to remove residual hemi-cellulose from the solid residue. The solid residue is preferably washed with fresh medium comprising acetic acid, more preferably in a counter flow or cross flow manner.

The washing liquid preferably comprises at least 50 vol. % water, preferably at least 70 vol. % water. In order to improve lignin removal, the washing liquid may comprise further acetic acid. Pure water or acetic acid/water mixtures are particularly suitable washing liquids. More than one washing steps may be carried out, for example a first washing step with a acetic acid/water mixture followed by one or more washing steps with water only or several washing steps with water only.

The washing step(s) may be typically carried out at a temperature in the range of from ambient to 100° C., preferably of from 20 to 90° C.

After each or all of the optional washing step(s), the obtained mixture preferably is again separated into a liquid stream and washed solid residue. The obtained washing liquid (washing black liquor) suitably comprises dissolved and hydrolysed hemicelluloses and acetic acid, and is preferably recycled at least in part to step (a), and partially sent to a work-up process to recover acetic acid as well as separate out the sugars from the hemicellulose.

Step (b) further preferably contains a second step wherein the water content of the solid residue is further reduced. The first separation yields typically a solids content of about 50 wt % However, in order to increase the effectiveness of the optional washing steps, the solids content preferably is increased to at least 65 wt %, preferably more than 65 wt %, more preferably at least 70 wt % This is preferably done by the use of an air blower or dryer. This permits more efficient washing steps while reducing the amount of water required.

The solid residue obtained in step (b) is sent to a second pulping stage in step (c).

In step (c), the solid residue is contacted with a pulping solution comprising peracetic acid to remove lignin.

Preferably, the solvent contains from 5 to 20 wt % of peracetic acid. More preferably, the solvent comprises at least 6 wt %, more preferably at least 7 wt %, yet more preferably 8 wt %, and yet more preferably 9 wt % of peracetic acid. Preferably, the solvent comprises less than 15 wt %, more preferably less than 13 wt % of peracetic acid.

Other constituents of the pulping solvent can comprise acetic acid, sulphuric acid and water.

Step (c) is executed at a temperature of from 50 to 95° C., more preferably from 65 to 85° C., most preferably at from 70 to 75° C. due to the higher activity of the peroxide in this temperature range, without significant loss due to disproportionation of the peracetic acid.

Preferably, step (c) is carried out at a pressure in the range of from 1 to 10 bar, more preferably of from 1 to 3 bar. Pre-treatment at atmospheric pressure is particularly preferred.

In step (d), the mixture obtained in step (c) is separated again into a second solid residue comprising delignified cellulose and a liquid phase comprising dissolved lignin. Similar to step (b), step (d) may further comprise one or more washing and separation steps to remove residual lignin from the solid residue. Steps (c) and (d) may be integrated, preferably in a continuous or batch wise process. Both steps may happen simultaneously, e.g. in a screw press.

Similarly to steps (a) and (b), the solid residue is preferably washed with solvent comprising peracetic acid, in a counter flow or cross flow manner. After the optional peracetic acid washing step, the obtained mixture may again be separated. The obtained liquid stream (also called black liquor) preferably is recycled at least in part to steps (c), and/or (a). The obtained solid residue is sent to an optional water-washing step.

While at least part of the black liquor obtained in steps (b) and (d) may be recycled to steps (a) and/or (c) to be used in the pulping solution, the remainder advantageously is sent to an acetic acid recovery process. This recovery process is preferably performed in a series of recovery units, of at least two units (and thus two steps, step (i) and step (ii)).

In step (i), in a first acetic acid recovery unit, the black liquor is preferably distilled by a flash distillation, leading to a liquid stream comprising lignin, also referred to as concentrated black liquor, and a vapour phase comprising acetic acid.

The vapour phase may be separated into an aqueous stream preferably containing of from 5 to 15 wt %, more preferably from 8 to 12 wt % acetic acid, and a second stream comprising more than 50 wt % of acetic acid, more preferably at least 55 wt % acetic acid. The first acetic acid stream may be sent to a second acetic acid recovery unit for step (ii), wherein the stream is separated into a bottom stream comprising more than 99 wt % of water, and a vapour phase comprising more than 50 wt % of acetic acid, respectively, by e.g. distillation or any other suitable separation method, including membrane separation.

The second stream containing more than 50 wt % acetic acid may preferably be condensed and at least in part recycled to step (a); it may preferably be directed in part to a lignin precipitation step; it may preferably be directed in part to an acetic acid washing unit; and/or in part may be sent to a rectifying column to obtain rectified acetic acid in a concentration of at least 90 wt %, preferably 95 wt %, which advantageously may be employed for a peracetic acid preparation step. The ratio of the above distribution will be selected by the skilled practitioner in order to keep the mass balance as correct as possible, as well as to keep energy use and use of new chemicals as low as possible. The stream comprising more than 99 wt % water may advantageously be recycled to washing steps in (b) and (d).

The process further preferably comprises an additional reaction step (iii) for the preparation of peracetic acid. This step comprises contacting a stream comprising acetic acid with an oxidative component, preferably hydrogen peroxide, to obtain a stream comprising peracetic acid, acetic acid and water. This stream may be employed for step (c). The reaction is preferably performed in the presence of suitable catalyst, more preferably in the presence of sulphuric acid.

Advantageously, the peracetic acid can be produced in step (iii) by contacting a stream comprising acetic acid and a stream comprising hydrogen peroxide in an aqueous reaction medium in the presence of a catalyst, preferably by feeding the streams continuously. The sulphuric acid catalyst preferably is recycled with the liquid stream separated off in step (b). Preferably, the acetic acid stream is at least in part formed by the rectified acetic acid.

The concentrated black liquor from the first acetic acid recovery unit preferably may be sent to the lignin recovery step, wherein lignin may be precipitated, preferably by adding water to the liquid fraction obtained in step (d). This allows most sugars left in this stream to remain in solution (further referred to as “syrup”). The water washing black liquor may be sent to the second acetic acid recovery unit for recovering acetic acid in step (ii), or it may be treated as wastewater. When the process reached steady state, the amount of acetic acid which should be made up will preferably be equal to that lost in the process, through in syrup and waste water streams. About 2 wt % of acetic acid are preferably continuously produced from the biomass intake due to hydrolysed acetate functionality in the lignocellulose.

In step (e) lignin is optionally precipitated from the liquid stream obtained in step (d). This may be done by any method that reduces the solubility of the dissolved lignin in the liquid phase obtained in step (d) as well as in the concentrated black liquor, such as distillation, addition of water, neutralisation of the acids or any other method known to the skilled person. The thus obtained lignin, optionally, after an additional washing step, was found to be of such quality that the material could be directly employed as a starting material for thermosetting resins.

In a preferred embodiment, the process further comprises a step (f) of subjecting the solid residue comprising delignified cellulose obtained in step (d) to an enzymatic hydrolysis to obtain a sugar solution and a solid residue.

In another preferred embodiment, the process of the invention does not require step (e), and comprises a process for the extraction of sugars from lignocellulose-comprising solid biomass, comprising the steps of:

(a) contacting the lignocellulose-comprising solid biomass with a pulping solution comprising at least 5 to 95% by weight of acetic acid at a temperature in the range of from 80 to 195° C.,
(b) separating the mixture obtained in (a) into a pre-treated solid residue and a liquid stream comprising dissolved hemicellulose;
(c) contacting the pre-treated solid residue with a pulping solution comprising at least 5 to 95% by weight of peracetic acid at a temperature in the range of from 50 to 95° C.,
(d) separating the mixture obtained in (c) into a solid residue comprising delignified cellulose, and a liquid stream comprising dissolved lignin, and
(f) subjecting the solid residue comprising delignified cellulose obtained in step (d) to an enzymatic hydrolysis to obtain a sugar solution and a solid residue.

Prior to being subjected to enzymatic hydrolysis in step (f), the (optionally washed) solid residue may advantageously be neutralised by washing the (optionally washed) residue with an aqueous alkaline solution, preferably followed by washing with water to remove the salts formed. Any aqueous solution of a water-soluble alkaline salt that forms soluble formate salts may suitably be used. Preferably a solution is used of an alkaline salt having a solubility in water of at least 0.1M at 20° C. Examples of preferred aqueous alkaline solutions are aqueous solutions of sodium hydroxide, potassium hydroxide, ammonia, or ammonium hydroxide.

The solid residue, optionally after neutralisation, is then preferably subjected to enzymatic hydrolysis to obtain monomeric sugars. Suitable enzymes and process conditions for enzymatic hydrolysis step (f) are known in the art, for example from WO2006/034590 or Lynd et al., Microbial Cellulose Utilization: Fundamentals and Biotechnology, Microbiology and Molecular Biology Reviews, 66 (2002), p 506-577.

For enzymatic hydrolysis step (f), suitably a slurry of the solid residue in water is made. If needed, the pH of the slurry is adjusted to a pH within the range of optimum pH for the enzymes used, typically in the range of from 4.0 to 6.0. Any suitable alkaline solution may be used for pH adjustment, for example a solution of sodium hydroxide, potassium hydroxide, calcium hydroxide, ammonia, or ammonium hydroxide. Preferably, the washing step in (d), optionally followed by washing with an alkaline solution, are carried out such that the slurry of the washed solid residue has a pH that is already within the range of optimum pH for the enzymes used. In that case, no pH adjustment is needed.

The pre-treated biomass can be subjected to enzymatic hydrolysis using cellulase enzymes, and optionally also hemicellulase enzymes, to obtain monomeric sugars.

Cellulase enzymes, optionally together with hemicellulase enzymes, can be added to the slurry of the washed solid residue, optionally after pH adjustment.

Typically, the cellulase enzyme dosage is in the range of from 5 to 50 Filter Paper Units (FPU) per gram of cellulose in the washed solid residue.

Enzymatic hydrolysis is typically carried out at a temperature in the range of from 30 to 70° C. for a period in the range of from 20 to 250 hours.

After enzymatic hydrolysis, the sugars obtained may be separated from the suspended solids using techniques known in the art, for example filtration or centrifugation.

The process may further comprise a further step, i.e. fermentation step (g), converting the sugar solution obtained in step (f) preferably with a suitable microorganism, preferably followed by a product recovery step (h). The products obtained may be alcohols, preferably ethanol or butanol, lipids or fatty alcohols; and/or chemical intermediates, e.g. lactic acid. Fermentation of sugars into ethanol, butanol lipids or fatty alcohols; and/or chemical intermediates, e.g. lactic acid is well-known in the art. Any such fermentation processes known in the art may be used for step (g).

Sugars recovered from the syrup, typically C5-sugars derived from hemicellulose hydrolysed in chemical pre-treating step (a), may be co-fermented in fermentation step (g), or fermented separately.

Products produced in fermentation step (g) may be recovered from the fermentation liquid by any suitable technique known in the art, usually by distillation. Ethanol or butanol product may be used in any suitable application, for example as gasoline component, while lipids or fatty alcohols may serve as feedstock for the preparation of e.g. diesel or kerosene fuels, preferably via hydrodeoxygenation, followed by hydroisomerisation of the thus obtained n-paraffins.

Alternatively, the sugars obtained in step (e) may also be employed in a thermochemical conversion, such as a aqueous phase reforming process for the conversion to hydrocarbons.

The invention will further be illustrated by the following, non-limiting examples.

### Example 1

Sugarcane bagasse was purchased in the Guanxi province in the south of China. It was ground and screened. The fraction that could not pass through a 20-mesh sieve was used in all pretreatment experiments. The composition of the sugarcane bagasse was determined according to corresponding Chinese standards, as shown in Table 1.

TABLE 1
Chemical composition of sugarcane bagasse and
corresponding test methods

| Items | Values | Methods |
|---|---|---|
| Moisture content | 3.42~6.07 | GB/T 2677.2- |
| (%, w/w) | | 1993 |
| Ash (%, w/w) | 1.38 | GB/T 2677.3- |
| | | 1993 |
| Hot water | 5.16 | GB/T 2677.4- |
| extractives (%, w/w) | | 1993 |
| 1% NaOH extractives | 34.20 | GB/T 2677.5- |
| (%, w/w) | | 1993 |
| Benzene-ethanol | 3.17 | GB/T 2677.6- |
| extractives (%, w/w) | | 1994 |
| Cellulose (%, w/w) | 44.98 | Nitric acid- |
| | | ethanol method |
| Holocellulose (%, | 76.76 | GB/T 2677.10- |
| w/w) ) | | 1995 |
| Klason lignin (%, | 18.45 | GB/T 2677.8- |
| w/w) | | 1994 |
| Acid-soluble lignin | 1.80 | GB/T 747-2003 |
| (%, w/w) | | |
| Total lignin (%, | 20.25 | GB/T 2677.8- |
| w/w) | | 1994, GB/T |
| | | 10337-1989 |

Preparation of Peracetic Acid: Peracetic Acid was prepared by reaction of acetic acid and 30% hydrogen peroxide, with an optimized volume ratio of 1.5:1 at room temperature for 72 h, and under addition of 1.5% (w/w) of sulfuric acid as a catalyst. The determination of peracetic acid concentration was in accordance with Chinese standard GB/T 19108-2003.
Pulping process: The acetic acid pretreatment was carried out in a 1000 ml glass flask with a heating jacket, equipped with a Teflon stirring paddle to help to homogenize the system and separate the fibers at a fixed rotation speed. In each batch experiment, 50 g sugarcane bagasse was packed into the flask and certain volume of acetic acid was added. The system was mixed as homogeneously as possible before being heated. The condition for the pretreatment were as follows: 55 wt % acetic acid concentration, 9:1 liquor ratio, 0.3 wt % sulfuric acid concentration, heated to and subsequently kept at the boiling point (103° C.) for 2.5 h. After pretreatment process the solid was extruded to release black liquor and then washed with 60% acetic acid at 5:1 liquor ratio (based on initial raw materials) and extruded again till the liquid content was about 75%. The solid was then packed into another 1000 ml glass flask immerged in a water bath for further oxidative delignification stage using peracetic acid concentration of 10 wt % (based on initial raw material) at 70-75° C. for 3 h. After the pulping process was finished, the pulp was extruded to recover the black liquor and then washed with water till neutrality was achieved.

The lignin was recovered from the black liquor. in the lignin recovery section, the black liquor was first condensed 9 fold before water was added. The amount of water used for precipitating lignin from the concentrated black liquor was 2 kg/kg black liquor, although the exact amount was found to be non-critical, since the water amount had no significant influences on lignin yield, provided it was higher than 1 g/g black liquor. The precipitated lignin was washed with water, and found to be of high quality.

Pulp analysis: The chemical compositions of the pulp was also determined in accordance with corresponding methods shown in Table 1. The Kappa number was measured according to TAPPI method T236m, and limiting viscosity (Degree of Polymerization, DP) according to ISO 5351. The optical property of the pulp was measured as brightness according to Chinese standard GB/T 7974-2002, using a WSB-3A brightness meter. Each data point represents the average of at least duplicate tests.
Paper sheets: Pulps were beaten to 50-55° SR in a Valley beater and handsheets of 60 g/m² basis weight were formed. The strength properties were tested according to corresponding Chinese standards as follows: folding endurance GB/T 475-1989, tear index GB/T 455.1-1989, and tensile strength and breaking length GB/T 453-1989.

### Analysis of Monosaccharide and Organic Acid

The main monosaccharides (glucose and xylose) and organic acid (acetic acid and formic acid) were determined by Shimadzu (Tokyo, Japan) HPLC using an Aminex HPX-87H column and RID-10A detector. The mobile phase was 0.005 M H₂SO₄ at a flow rate of 0.8 ml min-1.

Two alternative concentration ranges for the process according to the invention were illustrated:

Process 1 used a 65 wt % Acetic acid (AA) solvent to pretreat the bagasse at 100° C. with 0.3% H₂SO₄ as catalyst at 9:1 liquor ratio for 2.5 h, followed by the solid being washed with 65% AA solution and subsequently treated with 15 wt % peracetic acid (PAA) at 70-75° C. for 3 h. Process 2 uses a 55 wt % AA to pretreat the bagasse at boiling point (103° C.) with 0.3% H₂SO₄ as catalyst at 9:1 liquor ratio for 2.5 h, followed by the solid being washed with 60 wt % AA solution and subsequently treated with 10% PAA at 70-75° C. for 3 h. The process conditions led to similar pulping results.

To further study the effectiveness of the process according to the invention, a comparison is made with an Acetosolv pulping process, as shown in Table 2, using conditions as set out in Adilson R. Gonçalves, Denise S. Ruzene, Regina Y. Moriya, Luis R. M. Oliveria, 2005, Pulping of sugarcane bagasse and straw and biobleaching of the pulps: conditions parameters and recycling of enzymes, Presented at the 59th Appita Conference, Auckland, New Zealand, 16-19 May 2005.

Available from the web site http://www.tappsa.co.za/html/pulping_of_sugarcane_bagasse.html>.

TABLE 2
Comparison of the AA-PAA pulping process of
bagasse with other pulping processes

| | | | Comp.: |
|---|---|---|---|
| | Process 1 | Process 2 | Acetosolv |
| Pulp | 52.6 | 52.2 | 51.2 |
| Yield (%) | | | |
| Kappa | 16.4 | 22.0 | 41.5 |
| number K | | | |
| Selectivity | 3.26 | 2.37 | 1.23 |
| (Y/K) | | | |
| DP | 1138 | 1142 | 990 |
| ISO | 43.2 | 40.6 | 22.07 |
| brightness | | | |
| (%) | | | |
| Beating | ND | 60 | ND |
| degree | | | |
| Grammage | ND | 59.83 | ND |
| (g/m²) | | | |
| Tear | ND | 2.17 | ND |
| index | | | |
| (mN • m²/g) | | | |
| Folding | ND | 2 | ND |
| endurance | | | |
| Tensile | ND | 50.48 | ND |
| strength | | | |
| (N • m/g) | | | |

The process according to the invention was better than the acetosolv process for all the listed indexes.

### Example 2

Wheat straw was employed as substrate, using the conditions of process 1 set out above for the pretreatment, using different temperatures and liquor ratios (Table 3).

TABLE 3

| Temperature | | | |
|---|---|---|---|
| (° C.) | 100 | 105 | 105 |
| Liquor Ratio | 9:1 | 9:1 | 10:1 |
| Pulp Yield (%) | 56.4 | 46.1 | 43.8 |
| Kappa number K | 40.4 | 21.2 | 17.1 |
| Selectivity(Y/K) | 1.40 | 2.17 | 2.56 |
| DP | 1077 | 1021 | 981 |
| ISO brightness | 31.1 | 39.7 | 39.4 |
| (%) | | | |

A further comparison of AA-PAA pulping of wheat straw with the Acetosolv process was conducted as shown in Table 4. It was shown that Acetosolv pulping, although having a high pulp yield had a high kappa number, indicating a much lower selectivity (Table 4).

TABLE 4
Comparison of AA-PAA pulping of wheat straw

| | Comp: Acetosolv | Process 1 |
|---|---|---|
| Pulp yield Y (%) | 49.4 | 43.8 |
| Kappa number K | 69.0 | 17.1 |
| Selectivity (Y/K) | 0.72 | 2.56 |
| DP | 1040 | 981 |
| ISO brightness (%) | 30.62 | 39.4 |

What is claimed is:

1. A process for the extraction of sugars and/or lignin from lignocellulose-containing solid biomass, comprising the steps of:
(a) contacting the lignocellulose-containing solid biomass with a pulping solution comprising at least 5 to 95% by weight of acetic acid at a temperature in the range of from 80 to 195° C. thereby producing a first mixture;
(b) separating the first mixture obtained in (a) into a pre-treated solid residue and a liquid stream comprising dissolved hemicellulose;
(c) contacting the pre-treated solid residue with a pulping solution comprising at least 5 to 95% by weight of peracetic acid at a temperature in the range of from 50 to 95° C. thereby producing a second mixture; and
(d) separating the second mixture obtained in (c) into a solid residue comprising delignified cellulose, and a liquid stream comprising dissolved lignin.

2. The process of claim 1 further comprising step (e) precipitating lignin from the liquid stream obtained in step (d).

3. The process of claim 1 further comprising step (f) of subjecting the solid residue comprising delignified cellulose obtained in strep (d) to an enzymatic hydrolysis to obtain a sugar solution and a solid residue.

4. The process of claim 3 further comprising step (g) of fermenting the sugar solution obtained in step (f) with a suitable microorganism.

5. The process claim 1 wherein the lignocellulose-containing biomass comprises corn or soy stover, cereal straw, wood, grass, and/or bagasse.

6. The process of claim 1 further comprising neutralising the solid residue obtained in step (d) prior to subjecting the residue to enzymatic hydrolysis step (f) by washing the residue with an alkaline solution.

7. The process of claim 6 wherein the neutrazation is followed by washing with water.

8. The process of claim 6 wherein the alkaline solution is an aqueous solution of sodium hydroxide, potassium hydroxide, ammonia, or ammonium hydroxide.

9. The process claim 1 further comprising step (i) recovering acetic acid from the liquid phase obtained in step (b).

10. The process of claim 9 wherein the recovered acetic acid is further rectified.

11. The process of claim 10 further comprising a step (iii) wherein the rectified acetic acid is converted to peracetic acid.
